(19) United States
(12) Patent Application Publication
(10) Pub. No.: US 2023/0061214 A1
(43) Pub. Date: Mar. 2, 2023

(54) GUIDED ANALYSIS OF SINGLE CELL SEQUENCING DATA USING BULK SEQUENCING DATA

(71) Applicant: KONINKLIJKE PHILIPS N.V., EINDHOVEN (NL)

(72) Inventors: Jie WU, CAMBRIDGE, MA (US); Yee Him CHEUNG, BOSTON, MA (US)

(21) Appl. No.: 17/793,974

(22) PCT Filed: Jan. 13, 2021
(86) PCT No.: PCT/EP2021/050536

§ 371 Date: Jul. 20, 2022

Related U.S. Application Data
(60) Provisional application No. 62/963,250, filed on Jan. 20, 2020.

(51) Int. Cl. G16B 30/00; G16B 20/00; G16B 40/20

(52) U.S. Cl. CPC G16B 30/00; G16B 20/00; G16B 40/20

(57) ABSTRACT

A system (400) configured to generate a variant profile and a gene expression profile from a single cell sample, comprising: variant validation data and gene expression comparison data; single cell DNA sequencing data comprising a plurality of verified variants; single cell RNA sequencing data comprising a gene expression profile for the sample; a processor (420) configured to: (i) validate the identified variants using the variant validation data by: comparing the identified variant to the validation data; and assigning a validated classification status to the variant if the variant corresponds to the validation data; (ii) compare the obtained gene expression data to the obtained expression comparison data; and (iii) generate, based on the comparison and using a projection function, a final gene expression profile for the single cell sample; and a user interface (440) configured to provide a report comprising the identified variants and the generated final gene expression profile.

### FIELD OF THE DISCLOSURE

The present disclosure is directed generally to methods and systems for improving variant calling and gene expression estimation from single cell data.

### BACKGROUND

Single cell analysis is an emerging tool for profiling genomics, transcriptomics, and proteomics at higher resolutions. The immediate advantage of this relatively new technique is that it allows researchers to analyze heterogeneity between different cells. For example, tumors are generally heterogeneous even in the same tissue from the same patient. This single cell analysis allows researchers to investigate the differences between subclones of tumors to discover bio-markers and also insights into evolutions of the tumor cells.

While there are many advantages of single cell analysis, there are some major limitations. One of them is sparse and noisy signals due to limited materials and the nature of the technique. Single cell DNA-Seq (scDNA-Seq) generally suffers from bias in amplification of the limited amounts of DNA extracted, which results in uneven coverage along the genome. Additionally, calling mutations, especially single-nucleotide variants (SNVs), from single cell data is challenging due to the lower and uneven coverage of the genome.

Single cell RNA-Seq (scRNA-Seq) protocols such as Drop-Seq essentially sequence the 3′ ends of mRNAs. Dropouts are common in scRNA-Seq and the reads only cover the 3′ sites of mRNAs in most protocols. scRNA-Seq data are generally sparse and noisy due to the nature of the library preparation protocol and difficulty of managing RNAs at single cell resolution. Compared to traditional RNA-Seq protocols for a typical bulk sample, which normally amplifies whole transcripts, scRNA-Seq is more like digital gene expression (DGE) profiling with more dropouts. In addition, due the cost of sequencing thousands of cells, smaller number of reads are typically sequenced for each cell.

### SUMMARY OF THE DISCLOSURE

There is a continued need for methods and systems that generate a more accurate variant profile and/or gene expression profile from single cell data. The present disclosure is directed to inventive methods and systems for characterizing a plurality of variants from single cell data, and to inventive methods and systems for characterizing a gene expression profile from single cell data. Various embodiments and implementations herein are directed to a system and method that obtains variant validation data comprising variants from DNA sequencing data for a plurality of samples. A single cell sample is analyzed by identifying variants in DNA sequencing data obtained from the single cell. Each of the identified variants are analyzed by comparing the variant to the variant validation data and assigning a verified or validated classification status to the variant if the variant corresponds to the validation data. The system compiles the verified/validated identified variants to generate a report comprising characterized DNA sequence data for the single cell sample, and then provides the report via a user interface or other mechanism.

Various embodiments and implementations herein are also directed to a system and method that obtains gene expression comparison data comprising a plurality of gene expression profiles. A single cell sample is analyzed by obtaining gene expression data from the single cell. The system compares the obtained gene expression data to the obtained expression comparison data. Based on the comparison and using a projection function, the system generates a gene expression profile for the single cell sample. The system can then generate and provide a report comprising the generated gene expression profile for the single cell sample.

Generally, in one aspect, is a system for generating a variant profile and a gene expression profile from a single cell sample. The system includes: (i) variant validation data comprising a plurality of variants from DNA sequencing data; (ii) gene expression comparison data, comprising one or more gene expression profiles; (iii) single cell DNA sequencing data, utilized to identify a plurality of variants; (iv) single cell RNA sequencing data, utilized to generate a gene expression profile for the single cell sample; (v) a processor configured to: validate at least some of the plurality of identified variants using the variant validation data, comprising for each identified variant: comparing the identified variant to the validation data; and assigning a validated classification status to the variant if the variant corresponds to the validation data; compare the obtained gene expression data to the obtained expression comparison data; and generate, based on the comparison and using a projection function, a final gene expression profile for the single cell sample; and (vi) a user interface configured to provide a report comprising the identified variants assigned with a validated classification status and the generated final gene expression profile for the single cell sample.

According to an embodiment, the variant validation data comprises pooled DNA sequencing data obtained from each of a plurality of single cells from the same sample, verified variants obtained from bulk DNA sequencing data, and/or variant data obtained from a public or private database.

According to an embodiment, the gene expression comparison data comprises pooled gene expression obtained from each of a plurality of single cells from the same sample, a gene expression profile obtained from bulk RNA sequencing data, and/or a plurality of gene expression profiles obtained from a public or private database.

According to another aspect is a method for characterizing a DNA sequence of a single cell sample using a single cell analysis system. The method includes: (i) obtaining variant validation data, the variant validation data comprising a plurality of variants from DNA sequencing data; (ii) obtaining DNA sequencing data for the single cell sample; (iii) identifying, from DNA sequencing data, a plurality of variants in the DNA sequencing data; (iv) validating at least some of the plurality of identified variants using the obtained variant validation data, comprising for each identified variant: comparing the identified variant to the validation data; and assigning a validated classification status to the variant if the variant corresponds to the validation data; and (v) compiling at least those identified variants assigned with a validated classification status to generate a report comprising characterized DNA sequence for the single cell sample, and providing the report.

According to an embodiment, the method further includes assigning an unvalidated classification status to the variant if the variant does not correspond to the validation data, and wherein the report comprises one or more unvalidated variants.

According to an embodiment, the classification status comprises a validation confidence level.

According to an embodiment, the step of identifying a plurality of variants in the DNA sequencing data comprises guided variant calling using the variant validation data.

According to an embodiment, the single cell analysis system comprises a machine learning algorithm configured to validate variants identified in the DNA sequencing data, wherein the machine learning algorithm is trained using the variant validation data.

According to another aspect is a method for generating a gene expression profile from a single cell sample using a single cell analysis system. The method includes: (i) obtaining gene expression comparison data, comprising a gene expression profile; (ii) obtaining gene expression data for the single cell sample; (iii) comparing the obtained gene expression data to the obtained expression comparison data; (iv) generating, based on the comparison and using a projection function, a final gene expression profile for the single cell sample; and (v) generating and providing a report comprising the generated gene expression profile for the single cell sample.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for generating a more accurate variant profile and/or gene expression profile from single cell data using a single cell analysis system, in accordance with an embodiment.

FIG. 2 is a flowchart of a method for generating a variant profile from single cell data, in accordance with an embodiment.

FIG. 3 is a flowchart of a method for generating a gene expression profile from single cell data, in accordance with an embodiment.

FIG. 4 is a schematic representation of a system for generating a more accurate variant profile and/or gene expression profile from single cell data, in accordance with an embodiment.

### DETAILED DESCRIPTION OF EMBODIMENTS

The present disclosure describes various embodiments of a system and method to improve the analysis of variants from DNA sequencing data obtained from a single cell, and to improve the generation of a gene expression profile from RNA sequencing data obtained from a single cell. More generally, Applicant has recognized and appreciated that it would be beneficial to provide a method that combines bulk sequencing data with single cell data to enhance variant calling and gene expression estimation from single cell data. To improve DNA variant calling, the system identifies variants in DNA sequencing data obtained from the single cell. The system also generates or gathers variant validation data comprising variants from DNA sequencing data for a plurality of samples. The variants identified from the single cell sequencing are analyzed by comparing each variant to the variant validation data and assigning a validated classification status to the variant if the variant corresponds to the validation data. The system compiles the validated identified variants to generate a report comprising characterized DNA sequence data for the single cell sample, and then provides the report via a user interface or other mechanism. Similarly, to improve the accuracy of a gene expression profile generated from single cell data, the system generates gene expression data from the single cell. The system also generates or obtains gene expression comparison data comprising a plurality of gene expression profiles. The gene expression profile generated for the single cell is analyzed by comparing the obtained gene expression data to the obtained gene expression comparison data. Based on the comparison and using a projection function, the system generates a gene expression profile for the single cell sample. The system then generates and provides a report comprising the generated gene expression profile for the single cell sample.

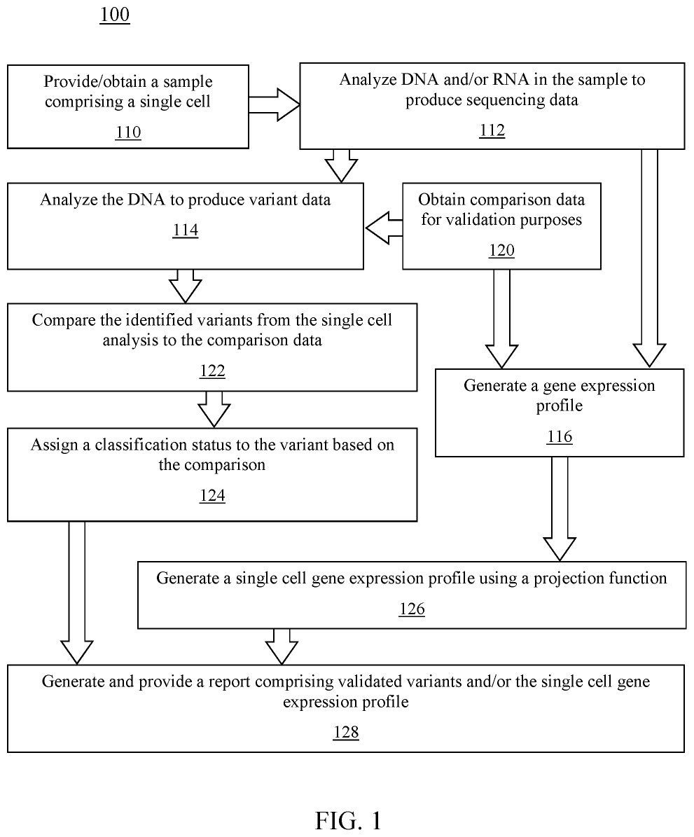

Referring to FIG. 1, in one embodiment, is a flowchart of a method 100 for generating a more accurate variant profile and/or gene expression profile from single cell data using a single cell analysis system. The single cell analysis system may be any of the systems described or otherwise envisioned herein, and may comprise any of the components described or otherwise envisioned herein.

At step 110 of the method, a sample is obtained from which one or more single cells will be analyzed. The sample may be any sample containing one or more cells for analysis, and may be obtained from any organism. For example, according to one embodiment the single cell is a tumor cell. A tumor sample may be any sample obtained from a patient's tumor, or from a tissue or location suspected to be or comprise a tumor. Tumor can be defined, for example, as a plurality of cancerous cells, and can be concentrated or diffuse. The tumor sample may be collected using any method or system for cell collection, such as through a biopsy or other tumor collection method. One or more single cells may be extracted from the tumor sample for single cell analysis.

At step 112 of the method, the single cell analysis system generates sequencing data from at least a portion of the genomic information of the sample, or otherwise receives sequencing data obtained from the sample. DNA and/or RNA is extracted from the cell obtained from the sample, and the genetic material is sequenced. The sequencing can be whole genome sequencing, whole exome sequencing, targeted exome sequencing, targeted SNP analysis, and/or any other type of sequencing. For DNA analysis, sequencing may be designed to enable variant identification. For RNA analysis, sequencing may be designed to enable generation of a gene expression profile.

According to an embodiment, the single cell analysis system comprises a sequencing platform configured to obtain sequencing data from the sample. The sequencing platform can be any sequencing platform, including but not limited to any systems described or otherwise envisioned herein. For example, the sequencing platform can be a real-time single-molecule sequencing platform, such as a pore-based sequencing platform, although many other sequencing platforms are possible. The sample and/or the nucleic acids therein may be prepared for sequencing using any method for preparation, which may be at least in part dependent upon the sequencing platform. According to an embodiment, the nucleic acids may be extracted, purified, and/or amplified, among many other preparations or treatments.

According to an embodiment, the single cell analysis system receives the sequencing data obtained by a sequencing platform from the sample. For example, the single cell analysis system may be in communication or otherwise receive the sequencing data from a local or remote sequencing platform which is separate from the single cell analysis system.

The DNA and/or RNA sequencing data may be utilized immediately for analysis as described or otherwise envisioned herein, and/or the sequencing data may be stored for later analysis. For example, the obtained sequencing data may be fed directly into the single cell analysis system for analysis, or may be stored locally or remotely, within or separate from the single cell analysis system, for later analysis. The generated and/or received sequencing data may be stored in a local or remote database for use by the single cell analysis system. For example, the single cell analysis system may comprise a database to store the sequencing data, and/or may be in communication with a database storing the sequencing data. These databases may be located with the single cell analysis system or may be located remote from the system, such as in cloud storage and/or other remote storage.

At step 114 of the method, the single cell analysis system analyzes the DNA sequencing data to identify variants within the single cell sample. The generated and/or received DNA sequencing data comprises a plurality of different variant types, including but not limited to single nucleotide variants, insertions, deletions, copy number variants, and gene fusions. Many other variant types are possible. Many different tools may be utilized to identify variants. For example, GATK is an example of one software package that may be utilized to identify variants. Gene fusions may be detected using a variety of systems, including but not limited to dRanger with Breakpointer, FusionMap, and/or other tools. Other structural variants such inversions, translocations, and others may be detected using a variety of systems, including but not limited to SVDetect, BreakDancer, and/or other tools.

At step 116 of the method, the single cell analysis system analyzes the RNA sequencing data to generate a gene expression profile for the single cell sample. The generated and/or received RNA sequencing data also comprises expression data, including but not limited to gene expression data, transcript expression data, exon expression data, splicing data, and/or allele-specific expression data. The expression data is obtained, analyzed, reported, and/or stored using any method utilized to do so from RNA sequencing data. The expression data can comprise information about allele-specific expression (ASE); allele-specific splicing (ASS); exon, transcript and gene (including long non-coding RNA, i.e. lncRNA) expressions; differential exon, transcript and gene (including lncRNA) expressions. Many different tools may be utilized to generate a gene expression profile for the single cell from the RNA sequencing data.

According to one embodiment, the tool utilized to identify variants in the scDNA-Seq data and/or scRNA-Seq data comprises a variant calling threshold or filter that may be adjusted. Due to the difficulty inherent in identifying variants from scDNA-Seq data and/or scRNA-Seq data, the threshold or filter for the tool may be lowered or otherwise modified. This may facilitate the identification of variants.

According to another embodiment, variants are identified in the scDNA-Seq data and/or scRNA-Seq data using more than just the single cell analysis. For example, according to one embodiment variants are identified from bulk sequencing data to obtain a high confidence list of variants, if bulk sequencing data for the relevant single cell is available. As another example, scDNA-Seq data and/or scRNA-Seq data from two or more relevant single cells, usually from the same population, may be merged and the merged data may be used to identify variants. Variant lists may be combined from two or more sources to generate a more comprehensive variant list. For example, the variant lists from bulk sequencing data, two or more single cells, and/or other sources may be combined for a comprehensive variant list.

At step 120 of the method, the single cell analysis system obtains comparison data for variant calling and/or validation purposes. For DNA analysis, the single cell analysis system obtains or receives variant validation data, comprising a plurality of variants from DNA sequencing data for a plurality of samples. Validated variants can be collected from public or private databases such as dbSNP and ClinVar, or can be derived from high quality public or private datasets such as TCGA, GIAB, etc. According to an embodiment and as described or otherwise envisioned herein, this comparison data can serve as a reference and/or can be used as additional data to enhance the quality of variant callings in the single cell analysis.

According to another embodiment, the comparison data obtained for validation purposes comprises bulk sequencing data from the same or a similar sample source. This bulk sequencing data may be obtained from another source, or may be obtained by bulk sequencing from the same sample from which a single cell is analyzed. As another example, the comparison data obtained for validation purposes comprises pooled data from two or more single cell analyses. Two or more single cells may be analyzed from the same or a similar sample source, and the variants from those two or more single cell analyses may be combined.

For RNA analysis, the single cell analysis system obtains or receives gene expression comparison data comprising a plurality of gene expression profiles. Gene expression profiles can be collected from public or private databases, such as CCLE, or computed from high quality public or private gene expression profiling datasets for similar samples. According to an embodiment and as described or otherwise envisioned herein, this comparison data can serve as a reference and/or can be used as additional data to enhance gene expression estimation for the single cell analysis.

According to an embodiment, the comparison data collected and/or identified in step 120 may be utilized to assist with variant calling in step 114 of the method and/or gene expression profile generation in step 116 of the method. For example, the comparison data may comprise one or more samples similar to the single cell sample, and thus the variants identified in these one or more samples may be more relevant to the variants in the single cell sample and can be utilized by the calling tools to improve calling in in the single cell sample.

At step 122 of the method, the identified variants from the single cell analysis are compared to comparison data for validation. According to an embodiment, the result of the comparison validates or rejects one or more identified variants from the single cell sample. The identified variants from the single cell analysis can be compared to comparison data using any method described or otherwise envisioned herein.

For DNA analysis, the single cell analysis system compares one or more of the identified variants to the validation data, and assigns a variant classification status to the variant. For example, at step 124 of the method, the system may assign a validated classification status to the variant if the variant corresponds to the validation data. The variant may correspond to the validation data if the variant is found in both the single cell analysis and in the comparison data which may be the obtained variant database data, the bulk sequencing data, and/or a pool of single cell analyses, among other sources. The variant classification status can comprise a wide variety of statuses. For example, the status may be identifiers such as likely validated, not validated, high confidence, low confidence, and many more. Additionally, classifying a variant as validated or a similar status can include a status less then definitively validated. Many identifiers, classifiers, and labels are possible.

According to an embodiment, the single cell analysis system may perform the comparison and classification status assignment using one or more operating parameters that facilitate the analysis. For example, the system may operate under the hypothesis or conclusion that the bulk sequencing data can represent a pool of single cells. Alternatively or additionally, the system may operate under the hypothesis or conclusion that a subset of single cells obtained from the same sample, typically in close proximity, will share the same variants. Alternatively or additionally, the system may operate under the hypothesis or conclusion that a majority of variants called from bulk sequencing data should present in at least a few (n) single cells, although of course there are exceptions and private CNVs and other variants can be found in single cells. These operating parameters can significantly facilitate the analysis of variants identified in the single cell analysis.

According to an embodiment, the single cell analysis system can assign a variant classification status to the variant based on the comparison using a variety of different approaches, which may be utilized individually or in any number of different combinations. For example, the system may compare a variant identified in the single cell analysis to the comparison data comprising variants identified in bulk sequencing and/or pooled data, and if the single cell variant is found in the bulk sequencing and/or pooled data it can be given a variant classification status such as validated or verified, or may be labeled or otherwise identified accordingly, or given a confidence score indicating a high confidence of accuracy or validation. Similarly, if the single cell variant is not found in the bulk sequencing and/or pooled data it can be given a variant classification status such as unverified or unvalidated, or may be labeled or otherwise identified accordingly, or given a confidence score indicating a low or no confidence of accuracy or verification/validation. As another example, rather than comparing variants, the system can directly validate the variants called from bulk or pooled sequencing data in single cells, which increases the sensitivity in single cell data.

According to another embodiment, machine learning algorithms can be used to learn the properties of true variants, such as high confidence variants in bulk or pooled data, and false variants, such as variants that only present in very small number of cells. Using this training data, the machine learning algorithm can then generate a model to classify variants identified in the single cell analysis as true and false. Of course, the identifiers ‘true’ and ‘false’ can be any identifier such as validated and unvalidated, high and low confidence, and many more. The machine learning algorithm can use any of a wide variety of features, including but not limited to base quality, position of the base in the read, location of the variant, nucleotide change type, depth at the position, variant frequencies, and many more.

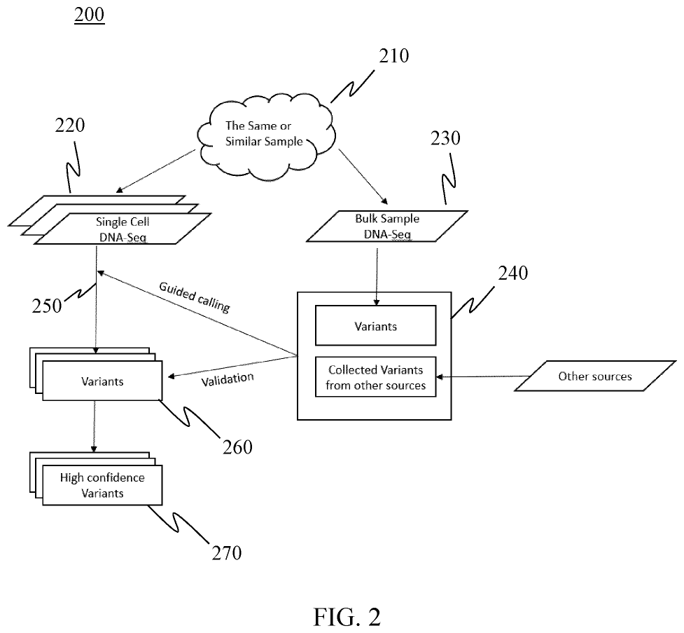

Referring to FIG. 2 is a flowchart showing one possible embodiment of a method 200 for DNA variant analysis using the single cell analysis system. At step 210, one or more samples are obtained for analysis and/or verification/validation. At 220, a single cell is analyzed to generate scDNA-seq data. At 230, bulk sequencing is performed on at least a portion of the sample to generate bulk DNA-seq data. At 240 the bulk DNA-seq data is analyzed to identify high-quality variants. This step may also comprise variant data from additional public or private sources, such as from public or private databases, and/or from pooled single cell analyses, among other possible sources. At 250, the identified high-quality variants are utilized to guide variant calling from the scDNA-seq data as described or otherwise envisioned herein. Similarly, at step 260, the identified high-quality variants are utilized to verify or validate variants identified in the scDNA-seq data as described or otherwise envisioned herein. At 270, the validated, high confidence, or otherwise similarly labeled variants from the scDNA-seq data are compiled.

Some or all of the variants and/or variant labels or classifications may be utilized immediately for an analysis or to generate a report as described or otherwise envisioned herein, and/or the data may be stored for later analysis. For example, the variants and/or variant labels or classifications may be stored locally or remotely, within or separate from the single cell analysis system, for later analysis. For example, the single cell analysis system may comprise a database to store the variants and/or variant labels or classifications, and/or may be in communication with a database storing the data.

For RNA analysis, the single cell analysis system compares gene expression data generated from the scRNA-Seq data of the single cell analysis to the comparison data, and generates a single cell gene expression profile. For example, at step 126 of the method, the system utilizes a projection function ƒ′, as described or otherwise envisioned herein, to generate the single cell gene expression profile.

According to an embodiment, the different protocols of single cell and bulk RNA-Seq must be considered or accounted for by the system when comparing scRNA-Seq data of the single cell with bulk sequencing data. Because of the different protocols, scRNA-Seq data from the sample would not result in a gene expression profile identical to the one obtained from bulk sequencing data from the same sample. Accordingly, it is necessary to bridge the gap between the scRNA-Seq data and the bulk sequencing data (or other comparison data). As described or otherwise envisioned herein, a function can be developed to project the gene expression profile from single cell data to a profile comparable to the profiles from bulk sequencing data.

According to an embodiment, the system comprises scRNA-Seq data sequenced from n cells from the same sample S. For each cell i, there is a gene expression profile eᵢ. Additionally, there is bulk RNA-Seq data for the same sample from which is obtained expression profile E_{b}. There is the following relationship between eᵢ and E_{b}:

E_{b}−ƒ(Sᵢ₌₁ⁿ(eᵢ)) (Eq. 1)

where S is a function to sum up the expression values from single cells, and ƒ is the projection function to project gene expression profile from single cell to bulk sample. The projection function can be fitted using E_{b} and Sᵢ₌₁ⁿ (eᵢ), then this function can be used to project individual single cell expression profile eᵢ to a profile which is comparable to a bulk sequencing gene expression profile Eᵢ:

Eᵢ=ƒ(eᵢ) (Eq. 2)

Similarly, a projection function ƒ′ to convert gene expression profile from bulk sample to single cell data can also be defined as:

eᵢ=ƒ′(Eᵢ) (Eq. 3)

Thus, the profiles from either technology can be converted to a comparable profile in the other technology.

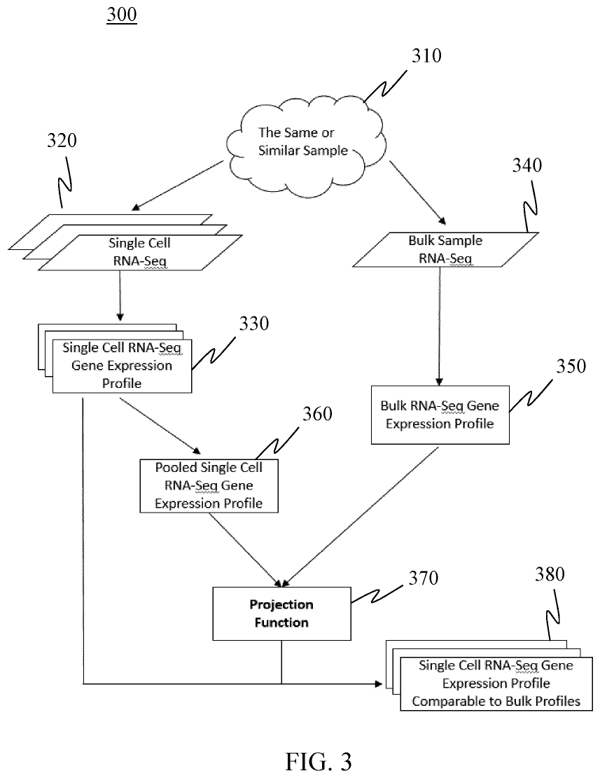

Referring to FIG. 3 is a flowchart showing one possible embodiment of a method 300 for RNA gene expression profile generation using the single cell analysis system. At step 310, one or more samples are obtained for gene expression profile generation. At 320, a single cell is analyzed to generate scRNA-seq data, and at 330 that scRNA-seq data is utilized to generate the single cell gene expression profile. At 340, bulk sequencing is performed on at least a portion of the sample to generate bulk RNA-seq data. At 350 the bulk RNA-seq data is analyzed to generate a bulk RNA-seq gene expression profile. This step may also gene expression profiles from additional public or private sources, such as from public or private databases, and/or from pooled single cell analyses such as that shown at step 360, among other possible sources. At 370, the bulk RNA-seq gene expression profile and/or the pooled single cell gene expression profile is utilized for the projection function, along with the single cell gene expression profile, as described or otherwise envisioned herein, to generate the final single cell gene expression profile comparable to bulk RNA-seq gene expression profiles.

The final single cell gene expression profile may be utilized immediately for an analysis or to generate a report as described or otherwise envisioned herein, and/or the data may be stored for later analysis. For example, the single cell gene expression profile may be stored locally or remotely, within or separate from the single cell analysis system, for later analysis. For example, the single cell analysis system may comprise a database to store the single cell gene expression profile, and/or may be in communication with a database storing the data.

At step 128 of the method, the single cell analysis system generates and provides a report comprising the classified variants and/or the single cell gene expression profile. The report may comprise any of the data or information generated or obtained as described or otherwise envisioned herein. The report may be electronic or printed, and may be stored. For example, the report may comprise a text-based file or other format. The report may comprise a database which is searchable for a particular variant or gene. The report may be sortable or otherwise configured for organization to allow easy analysis and extraction of information.

According to an embodiment, the single cell analysis system may visually display information about one or more of the variants and characterized expression status on a screen or other display method. A clinician or researcher may only be interested in one or several variants, and thus the variant analysis system may be instructed or otherwise designed or programmed to only display information obtained for the one or several variants.

According to an embodiment, the report or information may be stored in temporary and/or long-term memory or other storage. Additionally and/or alternatively, the report or information may be communicated or otherwise transmitted to another system, recipient, process, device, and/or other local or remote location.

According to an embodiment, once the report or information is generated, it can be provided to a researcher, clinician, or other user to review and implement an action or response based on the provided information. For example, a researcher or clinician may utilize the information to mine for variants in and/or gene expression of the sample, such as a tumor of a patient or a research subject. The user may manually review the report to review all variants or gene expression information, or to identify specific variants or gene expression through filtering and ranking, or may use software or other methodology. Identifying variants and analyzing gene expression is an important aspect of disease research, disease diagnosis, and disease treatment. Accordingly a clinician may, for example, diagnose a genetic disorder or hypothesize the existence of a particular genetic disorder based on the output of the report. The clinician may additional or alternatively select a specific treatment based on the output of the report.

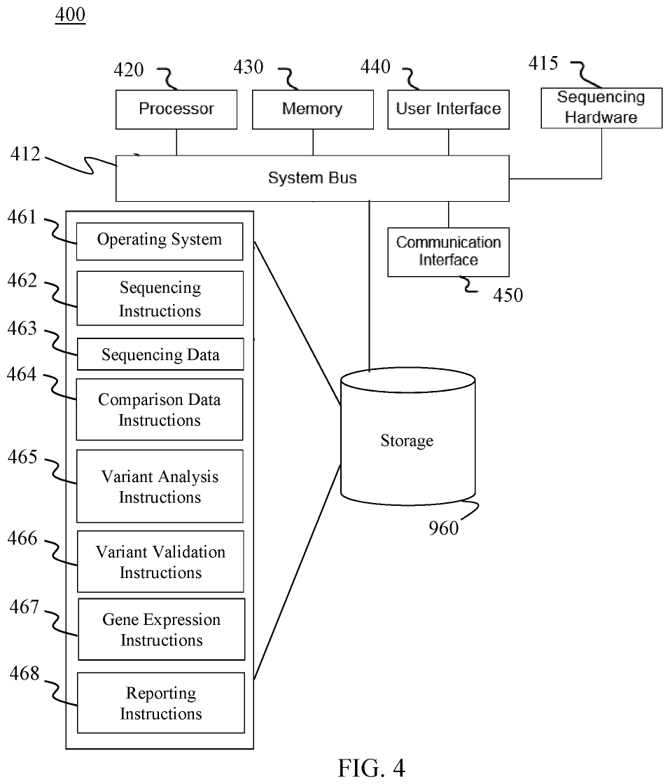

Referring to FIG. 4, in one embodiment, is a schematic representation of a single cell analysis system 400 configured to generate a more accurate variant profile and/or gene expression profile from single cell data using a single cell analysis system. The single cell analysis system 400 may be any of the systems described or otherwise envisioned herein, and may comprise any of the components described or otherwise envisioned herein.

According to an embodiment, system 400 comprises one or more of a processor 420, memory 430, user interface 440, communications interface 450, and storage 460, interconnected via one or more system buses 412. In some embodiments, such as those where the system comprises or directly implements a DNA and/or RNA sequencer or sequencing platform, the hardware may include additional sequencing hardware 415. It will be understood that FIG. 4 constitutes, in some respects, an abstraction and that the actual organization of the components of the system 400 may be different and more complex than illustrated.

According to an embodiment, system 400 comprises a processor 420 capable of executing instructions stored in memory 430 or storage 460 or otherwise processing data to, for example, perform one or more steps of the method. Processor 420 may be formed of one or multiple modules. Processor 420 may take any suitable form, including but not limited to a microprocessor, microcontroller, multiple microcontrollers, circuitry, field programmable gate array (FPGA), application-specific integrated circuit (ASIC), a single processor, or plural processors.

Memory 430 can take any suitable form, including a non-volatile memory and/or RAM. The memory 430 may include various memories such as, for example L1, L2, or L3 cache or system memory. As such, the memory 430 may include static random access memory (SRAM), dynamic RAM (DRAM), flash memory, read only memory (ROM), or other similar memory devices. The memory can store, among other things, an operating system. The RANI is used by the processor for the temporary storage of data. According to an embodiment, an operating system may contain code which, when executed by the processor, controls operation of one or more components of system 400. It will be apparent that, in embodiments where the processor implements one or more of the functions described herein in hardware, the software described as corresponding to such functionality in other embodiments may be omitted.

User interface 440 may include one or more devices for enabling communication with a user. The user interface can be any device or system that allows information to be conveyed and/or received, and may include a display, a mouse, and/or a keyboard for receiving user commands. In some embodiments, user interface 440 may include a command line interface or graphical user interface that may be presented to a remote terminal via communication interface 450. The user interface may be located with one or more other components of the system, or may located remote from the system and in communication via a wired and/or wireless communications network.

Communication interface 450 may include one or more devices for enabling communication with other hardware devices. For example, communication interface 450 may include a network interface card (MC) configured to communicate according to the Ethernet protocol. Additionally, communication interface 450 may implement a TCP/IP stack for communication according to the TCP/IP protocols. Various alternative or additional hardware or configurations for communication interface 450 will be apparent.

Storage 460 may include one or more machine-readable storage media such as read-only memory (ROM), random-access memory (RANI), magnetic disk storage media, optical storage media, flash-memory devices, or similar storage media. In various embodiments, storage 460 may store instructions for execution by processor 420 or data upon which processor 420 may operate. For example, storage 460 may store an operating system 461 for controlling various operations of system 400. Where system 400 implements a sequencer and includes sequencing hardware 415, storage 460 may include sequencing instructions 462 for operating the sequencing hardware 415, and sequencing data 463 obtained by the sequencing hardware 415, although sequencing data 463 may be obtained from a source other than an associated sequencing platform.

It will be apparent that various information described as stored in storage 460 may be additionally or alternatively stored in memory 430. In this respect, memory 430 may also be considered to constitute a storage device and storage 460 may be considered a memory. Various other arrangements will be apparent. Further, memory 430 and storage 460 may both be considered to be non-transitory machine-readable media. As used herein, the term non-transitory will be understood to exclude transitory signals but to include all forms of storage, including both volatile and non-volatile memories.

While single cell analysis system 400 is shown as including one of each described component, the various components may be duplicated in various embodiments. For example, processor 420 may include multiple microprocessors that are configured to independently execute the methods described herein or are configured to perform steps or subroutines of the methods described herein such that the multiple processors cooperate to achieve the functionality described herein. Further, where one or more components of system 400 is implemented in a cloud computing system, the various hardware components may belong to separate physical systems. For example, processor 420 may include a first processor in a first server and a second processor in a second server. Many other variations and configurations are possible.

According to an embodiment, storage 460 of single cell analysis system 400 may store one or more algorithms and/or instructions to carry out one or more functions or steps of the methods described or otherwise envisioned herein. For example, processor 420 may comprise comparison data instructions 464, variant analysis instructions 465, variant validation instructions 466, gene expression profile generation instructions 467, and/or reporting instruction 468.

According to an embodiment, comparison data instructions 464 direct the system to obtain comparison data for variant calling and/or validation purposes. The system may obtain the comparison data from any of a plurality of different possible sources. For example, for DNA analysis, the single cell analysis system can obtain or receive variant validation data, comprising a plurality of variants from DNA sequencing data for a plurality of samples. The variant data may also comprise bulk sequencing data, pooled single cell analysis data, variant data from one or more private or public sources, and/or from one or more other sources. For example, for RNA analysis, the single cell analysis system can obtain or receive gene expression comparison data comprising a plurality of gene expression profiles. The gene expression data may also comprise gene expression data from bulk sequencing, from pooled single cell analysis data, or gene expression data from one or more private or public sources, and/or from one or more other sources.

According to an embodiment, variant analysis instructions 465 direct the system to identify one or more variants in the DNA sequencing data from the single cell sample, and/or to analyze the RNA sequencing data to generate a gene expression profile for the single cell sample. Any method or tool may be utilized to identify variants or to generate the gene expression profile. According to one embodiment, the variant analysis instructions 465 direct the system to identify variants in the scDNA-Seq data and/or scRNA-Seq data using an adjustable variant calling threshold or filter. According to another embodiment, variant analysis instructions 465 direct the system to identify variants in the scDNA-Seq data and/or scRNA-Seq data using more than just the single cell analysis. For example, according to one embodiment variants are identified from bulk sequencing data to obtain a high confidence list of variants, if bulk sequencing data for the relevant single cell is available. As another example, scDNA-Seq data and/or scRNA-Seq data from two or more relevant single cells, usually from the same population, may be merged and the merged data may be used to identify variants. Variant lists may be combined from two or more sources to generate a more comprehensive variant list. For example, the variant lists from bulk sequencing data, two or more single cells, and/or other sources may be combined for a comprehensive variant list.

According to an embodiment, variant validation instructions 466 direct the system to validate one or more variants identified in the single cell data. For example, for scDNA-seq data, the identified variants from the single cell analysis are compared to comparison data for validation. According to an embodiment, the result of the comparison validates or rejects one or more identified variants from the single cell sample. For DNA analysis, the single cell analysis system compares one or more of the identified variants to the validation data, and assigns a variant classification status to the variant. For example, the system may assign a verified classification status to the variant if the variant corresponds to the validation data. The single cell analysis system can assign a variant classification status to the variant based on the comparison using a variety of different approaches, which may be utilized individually or in any number of different combinations. For example, the system may compare a variant identified in the single cell analysis to the comparison data comprising variants identified in bulk sequencing and/or pooled data, and if the single cell variant is found in the bulk sequencing and/or pooled data it can be given a variant classification status such as verified, or may be labeled or otherwise identified accordingly, or given a confidence score indicating a high confidence of accuracy or verification. Similarly, if the single cell variant is not found in the bulk sequencing and/or pooled data it can be given a variant classification status such as unverified, or may be labeled or otherwise identified accordingly, or given a confidence score indicating a low or no confidence of accuracy or verification. As another example, rather than comparing variants, the system can directly validate the variants called from bulk or pooled sequencing data in single cells, which increases the sensitivity in single cell data.

According to an embodiment, gene expression profile generation instructions 467 direct the system to compare gene expression data generated from the scRNA-Seq data of the single cell analysis to the comparison data, and to generate a single cell gene expression profile. For example, the system can utilize a projection function ƒ′, as described or otherwise envisioned herein, to generate the single cell gene expression profile. The projection function may utilize a bulk RNA-seq gene expression profile generated from bulk RNA-seq data. Similarly, the projection function may utilize gene expression profiles from additional public or private sources, such as from public or private databases, and/or from pooled single cell analyses, among other possible sources.

According to an embodiment, reporting instruction 468 direct the system to generate a user report comprising information about the analysis performed by the single cell analysis system. For example, the report may comprise any of the data or information generated or obtained as described or otherwise envisioned herein. The report may be electronic or printed, and may be stored. For example, the report may comprise a text-based file or other format. The report may comprise a database which is searchable for a particular variant or gene. The report may be sortable or otherwise configured for organization to allow easy analysis and extraction of information.

The reporting instruction 468 may direct the system to store the generated report or information in temporary and/or long-term memory or other storage. This may be local storage within system 400 or associated with system 400, or may be remote storage which received the report or information from or via system 400. Additionally and/or alternatively, the report or information may be communicated or otherwise transmitted to another system, recipient, process, device, and/or other local or remote location.

The reporting instruction 468 may direct the system to provide the generated report to a user or other system. For example, the single cell analysis system may visually display information about variants and/or gene expression on the user interface, which may be a screen or other display. A clinician or researcher may only be interested in one or several variants or genes, and thus the variant analysis system may be instructed or otherwise designed or programmed to only display information obtained for the one or several variants or genes.

According to an embodiment, the single cell analysis system and approach described or otherwise envisioned herein enables a researcher, clinician, or other user to more accurately determine the genotype and gene expression profile of the genetic sample, and thus to implement that information in research, diagnosis, treatment, and/or other decisions. This significantly improves the research, diagnosis, and/or treatment decisions of the researcher, clinician, or other user. This is especially important for cancer diagnosis, treatment, and research, which is one of the most common uses of single cell analysis.

Notably, the methods and systems described herein comprise different limitations each comprising and analyzing millions of pieces of information. For example, next-generation DNA sequencing data comprises reads that number in the 100 s of millions or even billions. Similarly, according to Illumina, “most [RNA-Seq studies) require 5-200 million reads per sample, depending on organism complexity and size.” Thus, identifying variants in scDNA-seq data and scRNA-seq data for the cell sample (as well as in the bulk, pooled, and/or otherwise obtained comparison data) will comprise millions or even billions of comparisons and calculations during alignment and variant calling. Similarly, comparison of the identified variants in the scDNA-seq and scRNA-seq data to the cell sample to the comparison data will comprise millions or even billions of comparisons and calculations. Just these steps alone, not counting others described or otherwise envisioned herein, comprise millions or billions of points of comparison, something the human mind is not equipped to perform, even with pen and pencil.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A single cell analysis system (400) configured to generate a variant profile and a gene expression profile from a single cell sample, comprising:
variant validation data comprising a plurality of variants from DNA sequencing data;
gene expression comparison data, comprising one or more gene expression profiles;
single cell DNA sequencing data, utilized to identify a plurality of variants;
single cell RNA sequencing data, utilized to generate a gene expression profile for the single cell sample;
a processor (420) configured to: (i) validate at least some of the plurality of identified variants using the variant validation data, comprising for each identified variant: comparing the identified variant to the validation data; and assigning a validated classification status to the variant if the variant corresponds to the validation data; (ii) compare the obtained gene expression data to the obtained expression comparison data; and (iii) generate, based on the comparison and using a projection function, a final gene expression profile for the single cell sample; and
a user interface (440) configured to provide a report comprising the identified variants assigned with a validated classification status and the generated final gene expression profile for the single cell sample.

2. The system of claim 1, wherein the variant validation data comprises pooled DNA sequencing data obtained from each of a plurality of single cells from the same sample, verified variants obtained from bulk DNA sequencing data, and/or variant data obtained from a public or private database.

3. The system of claim 1, wherein the gene expression comparison data comprises pooled gene expression obtained from each of a plurality of single cells from the same sample, a gene expression profile obtained from bulk RNA sequencing data, and/or a plurality of gene expression profiles obtained from a public or private database.

4. A method (100) for characterizing a DNA sequence of a single cell sample using a single cell analysis system (400), comprising:
obtaining (120) variant validation data, the variant validation data comprising a plurality of variants from DNA sequencing data;
obtaining (112) DNA sequencing data for the single cell sample;
identifying (114), from DNA sequencing data, a plurality of variants in the DNA sequencing data;
validating at least some of the plurality of identified variants using the obtained variant validation data, comprising for each identified variant: (i) comparing (122) the identified variant to the validation data; and (ii) assigning (124) a validated classification status to the variant if the variant corresponds to the validation data; and
compiling (128) at least those identified variants assigned with a validated classification status to generate a report comprising characterized DNA sequence for the single cell sample, and providing the report.

5. The method of claim 4, further comprising the step of assigning an unvalidated classification status to the variant if the variant does not correspond to the validation data, and wherein the report comprises one or more unvalidated variants.

6. The method of claim 4, wherein the classification status comprises a validation confidence level.

7. The method of claim 4, wherein the variant validation data comprises pooled DNA sequencing data obtained from each of a plurality of single cells from the same sample.

8. The method of claim 4, wherein the variant validation data comprises verified variants obtained from bulk DNA sequencing data.

9. The method of claim 4, wherein the variant validation data comprises variant data obtained from a public or private database.

10. The method of claim 4, wherein the step of identifying a plurality of variants in the DNA sequencing data comprises guided variant calling using the variant validation data.

11. The method of claim 4, wherein the single cell analysis system comprises a machine learning algorithm configured to validate variants identified in the DNA sequencing data, wherein the machine learning algorithm is trained using the variant validation data.

12. A method (100) for generating a gene expression profile from a single cell sample using a single cell analysis system (400), comprising:
obtaining (120) gene expression comparison data, comprising a gene expression profile;
obtaining (114) gene expression data for the single cell sample;
comparing (122) the obtained gene expression data to the obtained expression comparison data;
generating (126), based on the comparison and using a projection function, a final gene expression profile for the single cell sample; and
generating and providing (128) a report comprising the generated gene expression profile for the single cell sample.

13. The method of claim 12, wherein the gene expression comparison data comprises pooled gene expression obtained from each of a plurality of single cells from the same sample.

14. The method of claim 12, wherein the gene expression comparison data comprises a gene expression profile obtained from bulk RNA sequencing data.

15. The method of claim 12, wherein the gene expression comparison data comprises a plurality of gene expression profiles obtained from a public or private database.
